(19) United States
(12) Patent Application Publication
(10) Pub. No.: US 2011/0213227 A1
(43) Pub. Date: Sep. 1, 2011

(54) WIRELESS MEDICAL MONITORING SYSTEM

(76) Inventors: David Ziv, Kibbutz Baram (IL); Ilan Shopen, Rosh Pina (IL); Yosef Gandelman, Ashdod (IL); Avi Keren, Macabim (IL)

(21) Appl. No.: 13/061,550

(22) PCT Filed: Sep. 1, 2009
(86) PCT No.: PCT/IB09/53807

§ 371 Date: May 20, 2011

Related U.S. Application Data
(60) Provisional application No. 61/093,362, filed on Sep. 1, 2008.

(51) Int. Cl. A61B 5/1455

(52) U.S. Cl. 600/323

(57) ABSTRACT

A blood oxygen saturation level (SpO2) measurement subunit employed in a wireless transceiver unit connected to a medical monitor unit. An illumination emulator is used for emulating the characteristics of an illumination source of a pulse oximeter. The emulator utilities at least part of the energy coming from the SpO2 socket of the medical monitor. Energy originally intended to energize one illumination source of the pulse oximeter, energizes the power supply circuitry. A processor is employed for processing information about pulsing arterial blood of a patient received from a patient companion assembly (PCA). A digital to analogue converter is used for converting the PCA, to analogue signal. A low pass filter (LPF), filtering the signal to form a pulsative voltage signal represents the pulsing arterial blood of the patient, and is sent to the SpO2 socket of the medical monitor for displaying and further processing.

### FIELD OF THE INVENTION

The present invention relates to medical monitoring systems, more particularly to wireless medical monitoring systems.

### BACKGROUND OF THE INVENTION

The electrical activity of the heart can be recorded to assess changes over time or diagnose potential cardiac problems. Electrical impulses generated in the heart are conducted through body fluids to the skin, where they can be detected and printed out by a device known as an electrocardiograph. The printout is known as an electrocardiogram, or ECG. Typically, an ECG includes three distinguishable waves or components (known as deflection waves), each representing an important aspect of the cardiac function.

Blood pressure is the amount of force per unit area (pressure) that blood exerts on the walls of the blood vessels as it passes through them. There are two specific pressure states measurable for blood pressure: pressure while the heart is beating (known as systolic blood pressure) and pressure while it is relaxed (known as diastolic blood pressure). Diastolic blood pressure measures the pressure in the blood vessels between heartbeats, when the heart is resting. Automated devices can measure blood pressure with an inflatable cuff and an automated acoustic or pressure sensor that measure blood flow, employing a non-invasive blood pressure sensor. The sensor can be used to measure systolic and diastolic blood pressure.

Pulse oximetry is a non-invasive method used to measure blood oxygen saturation level (SpO₂) by monitoring the percentage of hemoglobin, which is saturated with oxygen; as well as measuring heart rate. A sensor is placed on a thin part of the patient's anatomy, usually a fingertip or earlobe, or in the case of a neonate, across a foot, and red and infrared light is passed from one side of the body part to the other. Changing absorbance of each of the wavelengths is measured, allowing determination of the absorbances due to the pulsing arterial blood alone, excluding venous blood, skin, bone, muscle and fat. Based upon the ratio of changing absorbance of the red and infrared light caused by the difference in color between oxygen-bound (bright red) and oxygen unbound (dark red) blood hemoglobin, a measure of oxygenation (the percentage of hemoglobin molecules to which oxygen molecules are bound) can be made.

A patient monitor usually is a device that includes a processor, display, keyboard, recorder, sensors and cables. It integrates the functions of measuring, recording and alarming, which are useful for patient status analysis and monitoring. The monitor can, inter alia, measure and record a patient's vital signs including ECG data, blood pressure, respiration, temperature, and SpO₂ in real time, such a monitor is widely used in many clinical sites such as the operating room, intensive care unit and so on.

WO08004205, the contents of which are incorporated herein by a reference, assigned to the owner of the present application, describes an operator-controllable medical monitoring system including one or more medical sensors that are adapted to monitor one or more patient characteristics. The monitoring system comprises a plurality of medical monitors, each including a wireless monitor transceiver, a medical information display and a patient companion assembly with a patient companion assembly wireless transceiver and a medical monitor selector. The monitor selector is wirelessly operable to initially select one of the plurality of medical monitors and to provide a monitor selection indication which is visually sensible to the operator.

### BRIEF DESCRIPTION OF THE DRAWINGS

The present invention will be understood and appreciated more fully from the following detailed description, taken in conjunction with the drawings in which:

FIG. 1 is a schematic depiction of the functional control of the framework in which the present invention is implemented;

FIG. 2 is a schematic depiction of the main modules and functional subunits of the of the framework in which the present invention is implemented;

FIG. 3 is a block diagram of an ECG subunit employed in a patient companion assembly wireless transceiver;

FIG. 4 is a detailed description block diagram of the PCAWT showing one channel route in accordance with a preferred embodiment of the present invention;

FIG. 5 is a simplified block diagram of the monitor ECG subunit in accordance with the present invention;

FIG. 6 is a schematic depiction of the SpO₂ subunit of the PCAWT;

FIG. 7 is a schematic depiction of the SpO₂ subunit of the monitor-side SpO₂ subunit;

FIG. 8 is a schematic block diagram of an LED emulator of SpO₂ subunit in accordance with some embodiments of the present invention;

FIG. 9 is an electronic scheme of LED emulator in accordance with some embodiments of the present invention;

FIG. 10 is an electronic schema of isolated continuous pulsative voltage to pulse light converter in accordance with some embodiments of the present invention;

FIG. 11A is an electronic schema of continuous voltage to pulse light converter LED off equivalent scheme;

FIG. 11B is an electronic scheme of continuous voltage to pulse light converter LED on equivalent scheme;

FIG. 12 is a schematic depiction of the monitor wireless transceiver module (MWT) employed in accordance with the present invention;

FIG. 13 is a schematic depiction of pressure sensor load emulator and current flow controller in accordance with some embodiments of the present inventions; and

FIG. 14 is an electronic scheme of medical thermistor emulator in accordance with the present invention.

The following detailed description of the invention refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION OF SOME EMBODIMENTS OF THE PRESENT INVENTION

The prior art system in which the present invention is implemented receives data from one or more sensors detecting physiological or medical parameters of one or more patients. The system includes one or more monitors, each monitor including a wireless monitor transceiver and a medical information display. The system further includes a patient companion assembly (PCA) which includes a dedicated wireless transceiver (PCAWT) and a monitor selector for selecting a specific monitor. Both the PCAWT and the monitor selector are operative to initially select one of the plurality of medical monitors and to provide a monitor selection indication which is visually sensible by the operator.

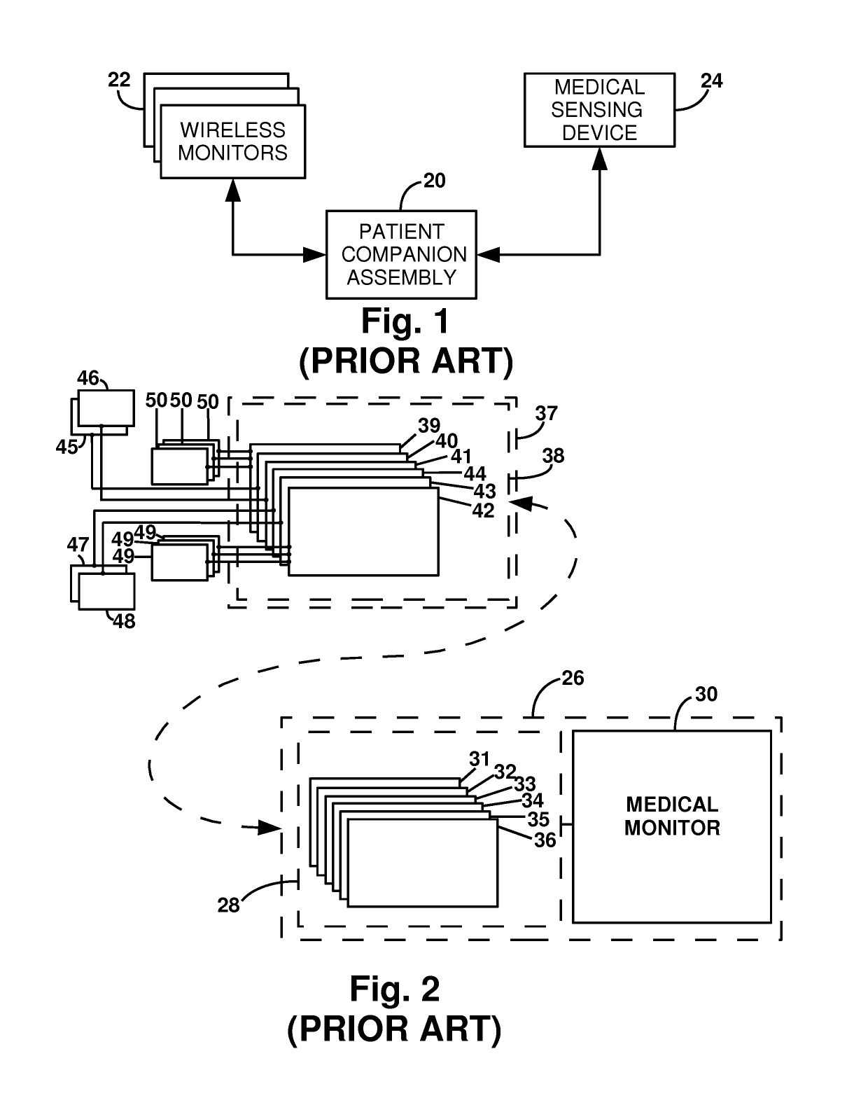

A schematic description of the functional control of the prior art framework in which the present invention is implemented as described in FIG. 1 to which reference is now made. A patient companion assembly (PCA) 20, which includes a transceiver and a monitor selector, which selects one of a plurality of wireless monitors (WMs) 22. WM 22 communicates with one or more medical sensing devices 24 via a wireless transceiver associated with PCA 20. Examples of sensing devices applicable in the context of the system of the present invention are blood pressure sensors, ECG sensors, SpO₂ sensors, temperature sensors, respiratory and blood chemistry parameter sensors. A system of the invention is dependent on the functionality of the PCA 20 but, not all communications are necessarily established via such PCA.

The main modules and subunits of a prior art system in which the present invention is implemented are described in FIG. 2 to which reference is now made. Wireless medical monitor 26 includes two main units, wireless monitor transceiver unit (WMT) 28 and medical monitor unit 30. Monitor wireless transceiver (MWT) 28 includes several subunits which are used for processing information obtained from sensing devices that are applicable in the context of the system of the present invention, for example ECG subunit 31, SpO₂ subunit 32, temperature subunit 33, pressure subunit 34, respiratory subunit 35 and blood chemistry sub unit 36. In accordance with the present invention each of these subunits may share one or more components such as a wireless communication subsystem, a processor, a digital to analogue (D/A) converter, an analogue to digital (A/D) converter, opto-couplers, power supplies and multiplexers. The patient companion assembly (PCA) 37 includes wireless transceiver (PCAWT) 38. The PCAWT includes typically several subunits each of which used for processing information derived from sensing devices that are applicable in the context of the system of the present invention. For example ECG subunit 39, SpO₂ 40 subunit, temperature subunit 41, pressure subunit 42, respiratory subunit 43 and blood chemistry sub unit 44. These subunits typically refer each to a matching subunit in the MWT. In one embodiment of the present invention each of the PCAWT subunits may share one or more electrical components such as wireless communication subsystem a processor, a digital to analogue (D/A) converter, an analogue to digital (A/D) converter and multiplexers. Subunits SpO₂ 40, temperature 41, blood chemistry 44 and respiratory 43 are each connected to its respective sensors: SpO₂ 45, temperature 46, blood chemistry 47 and respiratory 48. Pressure subunit 42 is connected to one or more pressure sensors 49 and typically has one channel for each pressure sensor 49 for further processing. ECG subunit 39 is connected to one or more ECG sensor 50s and typically has one channel for each ECG sensor 50 for further processing.

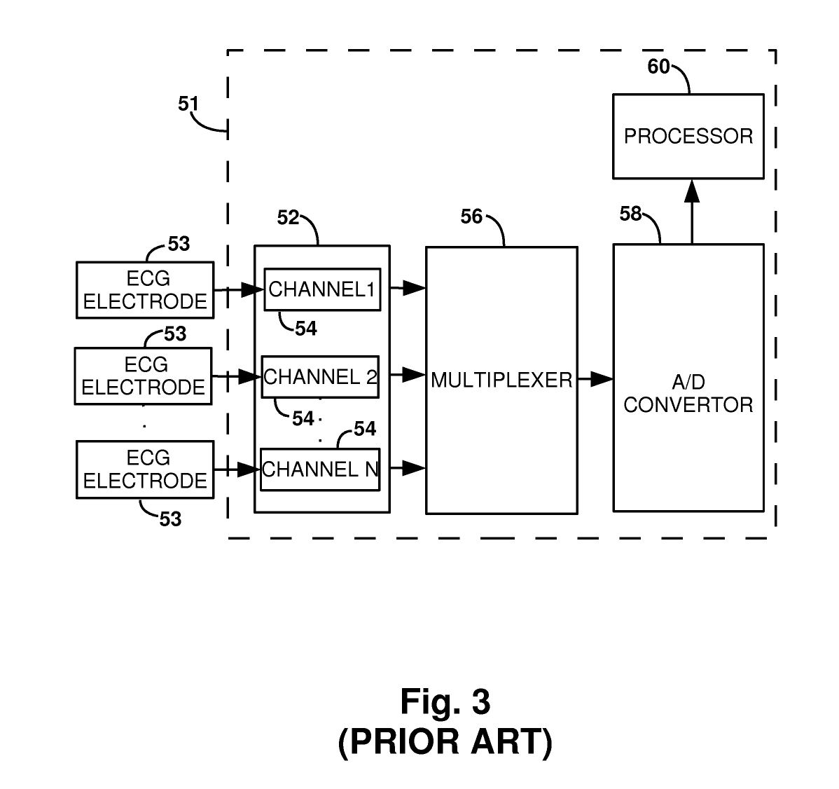

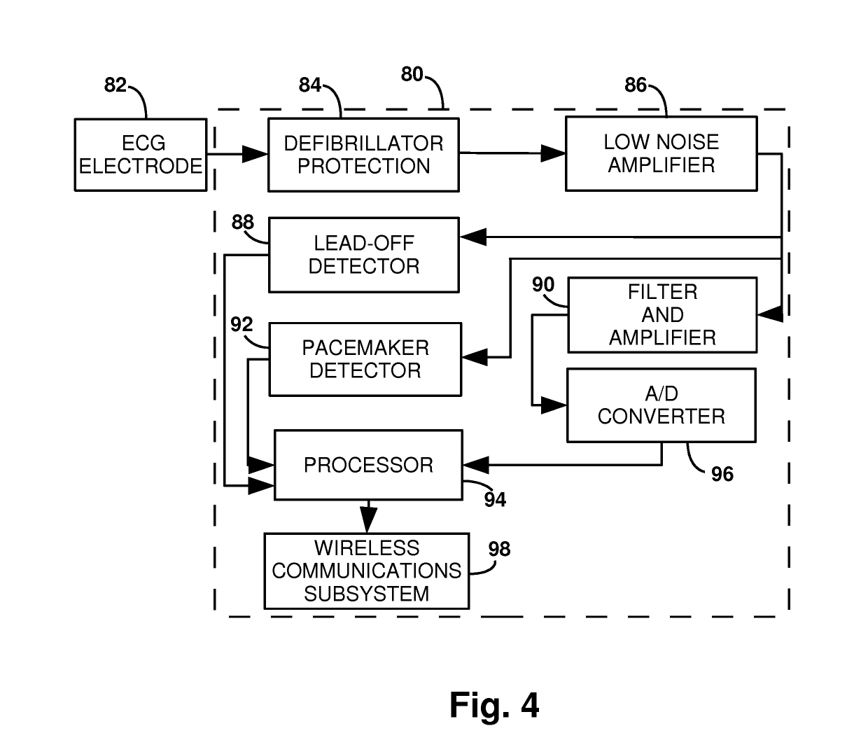

FIG. 3 shows a schematic prior art block diagram of an ECG subunit employed in the PCAWT. Such an ECG subunit 51 includes a medical sensor interface subunit 52, which, in this example, processes inputs from a plurality of ECG electrodes 53. Typically, medical sensor interface subunit 52 includes one or more ECG connectors, not shown and one or more channels. Each connecter is connected to a respective input ECG channel interface 54. ECG channel interface 54 includes an amplifier and a filter. The output signals from the ECG input channel each interface 54 are preferably supplied via a multiplexer 56 and an A/D converter 58 to an ECG input processor 60, which adapts the signals to digital wireless communications and supplies them to a digital wireless communications subsystem, not shown. A more detailed schematic description of the ECG subunit employed in the PCAWT demonstrating one channel route is described in FIG. 4 to which reference is now made. ECG subunit 80 processes input arriving from ECG electrode 82. The analogue signal of the ECG electrode is provided to defibrillator protection circuitry 84 which is an electrical circuit designed to withstand a high-voltage burst from a defibrillator. The defibrillator output signal is amplified by preamplifier 86 which is preferably a low noise amplifier (LNA). The preamplifier output signal is provided to lead-off detector 88, and in parallel to both band pass filter and amplifier unit 90 and to pacemaker detector 92. Lead-off detector 88 is used to confirm the intactness of an ECG lead connection to a body of a patient. Preferably, the band-pass filter is in the frequency range of 0.05-300 Hz. The output pulse of the pacemaker is used for signaling processor 94 as to the presence of a pacemaker signal. The filter and amplifier output signal is converted to digital data by analogue to digital (A/D) 96 for further processing in processor 94. Referring again to FIG. 3 the outputs of channels 54 are multiplexed in two different sequences for example, in a first cycle, the sequence direction of channel selection is selected by the multiplexer from channel 1 to channel N and in a second cycle of multiplexing, the direction of channel selection is from channel N to channel 1. This sequencing approach is employed in order to compensate for a phase shift taking place between the sampled channels when using a single A/D. Referring again to FIG. 4, the ECG signals which are processed in processor 94, are adapted for digital wireless communications and are subsequently fed into the digital wireless communications subsystem (WSS) 98. In one aspect of the present invention the WSS can send to the wireless monitor data about one or more disconnected ECG leads. With such information, the wireless monitor can select which of the connected leads is the reference lead and send this information to the PCA transceiver. The ECG transceiver has a self test generator that injects pulses in order to test the entire path of the ECG data. The ECG transceiver further includes an electrical circuit for filtering out the frequency of the network power which is typically 50/60 [Hz].

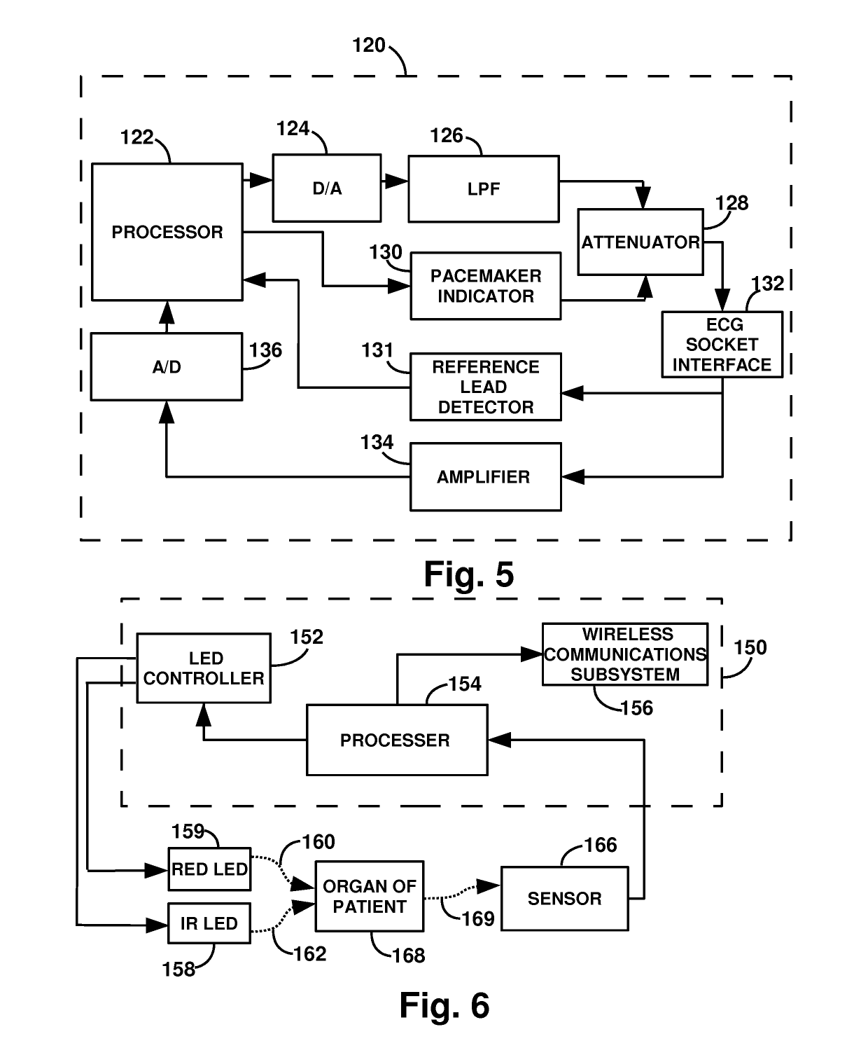

Reference is now made to FIG. 5 showing a simplified block diagram of the ECG subunit in the monitor in accordance with the present invention. ECG monitor subunit 120 includes processor 122 which processes the ECG data received from the PCAWT. The received ECG data typically includes one or more measurements for each ECG lead. The processed ECG data is provided to D/A 124 and than filtered preferably by a low pass filter (LPF) 126. The signals from the filter are attenuated by attenuator 128 for adapting the signals to the desired intensity levels acceptable by the medical monitor as input. Attenuator 128 attenuates the signal arriving from pacemaker indicator 130 too. The flow of output signals from the attenuator, can be stopped before reaching ECG socket interface 132, by a switcher, not shown, in case that an ECG was disconnected at the transmitter's end, for example as a result of an ECG being disconnected from a patient. ECG data provided to the monitor can also feedback-control the transceiver of the ECG monitor interface subunit. Some commercial medical monitors can decide which ECG lead is the reference lead, and in one aspect of the present invention this data is provided to the ECG monitor interface subunit transceiver for sending the data to the PCA transceiver through reference lead detector 131. Signals which are sent to ECG socket interface 132 are amplified by amplifier 134, converted to digital form by A/D 136 and verified by processor 122.

A schematic description of the SpO₂ subunit of the PCAWT of the MWT is described in FIG. 6 to which reference is now made. SpO₂ subunit of the PCAWT 150 includes LED controller 152, processor 154, and wireless communications subsystem 156. Led controller 152, which are controlled by processor 154 such power supplies are driving IR LED 158 and red LED 159. Processor 154 controls the radiation intensity of LEDs 158 and 159. The radiation of LEDs 158 and 159 are designated by dashed arrows 160 and 162 respectively. One or more sensors such as photo diode 166 are placed on an organ of the patient 168, such as a finger. Changes in the respective absorbances of each of the two wavelengths of the LEDs are measured. The radiation from the patient 168 is designated by dashed arrow 169. The measured LEDs analogue signals are filtered in the frequency ranges of the pulsing arterial blood and converted to digital, not shown. The digital data is provided to processor 154 for further processing. Information about the patient pulsing arterial blood is derived in processor 154 and is sent through wireless communications subsystem 156 to SpO₂ subunit 180 of the MWT.

A typical SpO₂ of a medical monitor, as in most standard medical monitors known in the art such as Hewlett Packard Merlin Multi-Parameter Monitor, supplies energy to the LEDs of the pulse-oximeter. In accordance with the present invention, the energy coming from a typical SpO₂ of a medical monitor otherwise originally intended to be supplied to energize the LEDs of the pulse-oximeter, is instead utilized for powering the internal power supplies of the SpO₂ subunit in the monitor-side.

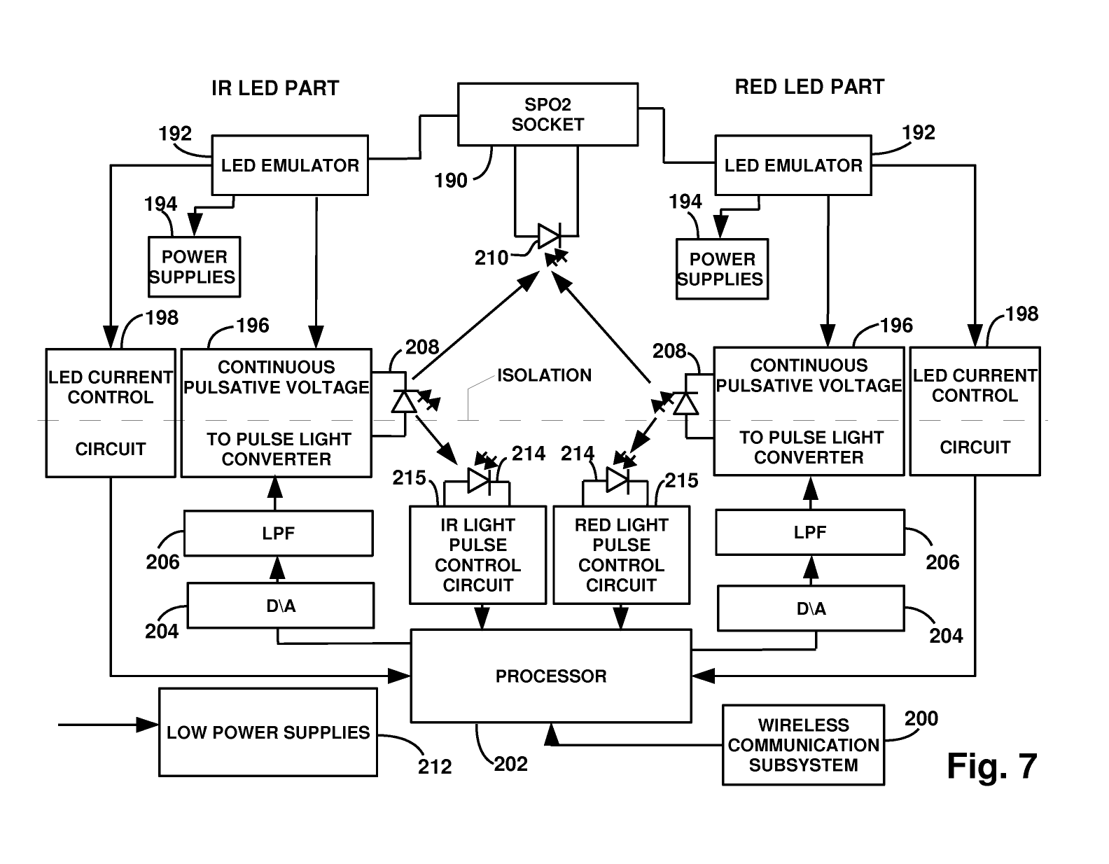

A schematic block diagram of the SpO₂ subunit of the monitor-side SPO2 subunit is described in FIG. 7. Subunit consists from two optically isolated parts: one part electrically connected to SpO₂ socket and the other part electrically connected to processor and to wireless communication subsystem. For each LED SpO₂ connected part includes the LED emulator, the power supplies and the commons for two parts optically isolated circuits which include LED current control circuit (LCC) and continuous pulsative voltage to pulse light converter circuit (CPPL).

Illumination emulator such as, LED emulators 192 are use to emulate the characteristics of a typical illumination source such as LED, with a typical forward voltage rating between 1 and 2.5 Volts of DC. A detailed description of LED emulators 192 will be given below in more detail. Led emulator 192 drives power supplies with voltage pulses. Power supplies 194 include, both not shown, a pulse to positive DC converter and a pulse to negative DC converter. LED emulator includes current divider, not shown, that is used to divide the electrical current coming from the SpO₂ sockets. Part of the input current of LED emulator 192 flows to continuous pulsative voltage to pulse light converter circuitry (CPPL) 196. The other part of the input current of LED emulator 192 flows to LED current control circuitry (LCC) 198. The part of the LED current pulses are converted to pulses of light in order to electrically isolate the SpO₂ socket from the processor. The LCC includes a photodiode and a light to voltage converter, not shown, for converting the light pulses to electrical pulses. The LCC further includes a low pass filter (LPF) and an analogue to digital converter (A/D) the digital data is sent to a processor, not shown, for further processing in order to measure the current pulses from SpO₂ socket 190 for purposes of correct control of the IR and red signal circuits.

The Information about the patient pulsing arterial blood is received from the PCA through wireless communications subsystem 200 and sent to processor 202 for further processing. The Information about the patient's pulsing arterial blood is converted to analogue signal by digital to analogue converter 204 and filtered through LPF. The out-put signal of LPF 206 is a pulsative voltage signal, meaning, a continuous electrical signal representing the pulsing arterial blood of the patient. CPPL 196 receives the pulses of current from LED emulator and the pulsative voltage. In the CPPL 196, the amplitude of the pulsative voltage signal, modulates the pulses of current from LED emulator 192. The light emitted from LEDs 208 is driven by the modulated pulses of LED emulator 192. Typically the frequency of electrical signal that drives the LEDs of a standard SpO₂ is in the ranges of 75 Hz to 10 kHz, thus the pulses of current from LED emulator 192 are also in the range of 75 Hz to 10 kHz. Photodiode 210 detects the modulated pulses of light emitted from LEDs 208. The light beams emitted from LEDs 208 are modulated signals of the detected radiation from the organ of a patient with the timing of the current pulses coming from SpO₂ socket 190. Low power supplies 212 circuitry is used to supply energy to one or more modules in the SpO₂ subunit. An energy storage unit, not shown and will described later in more detail energized low power supplies 212. In addition to photodiode 210, photodiodes 214 also detect the modulated pulses of light emitted from LEDs 208. Light pulse control circuits 215 and photodiodes 214 are used in association with processor 202 for insuring that the information about the patient's pulsing arterial blood sent to D/A 204 is the same as the information collected by the photodiode 210 respectively.

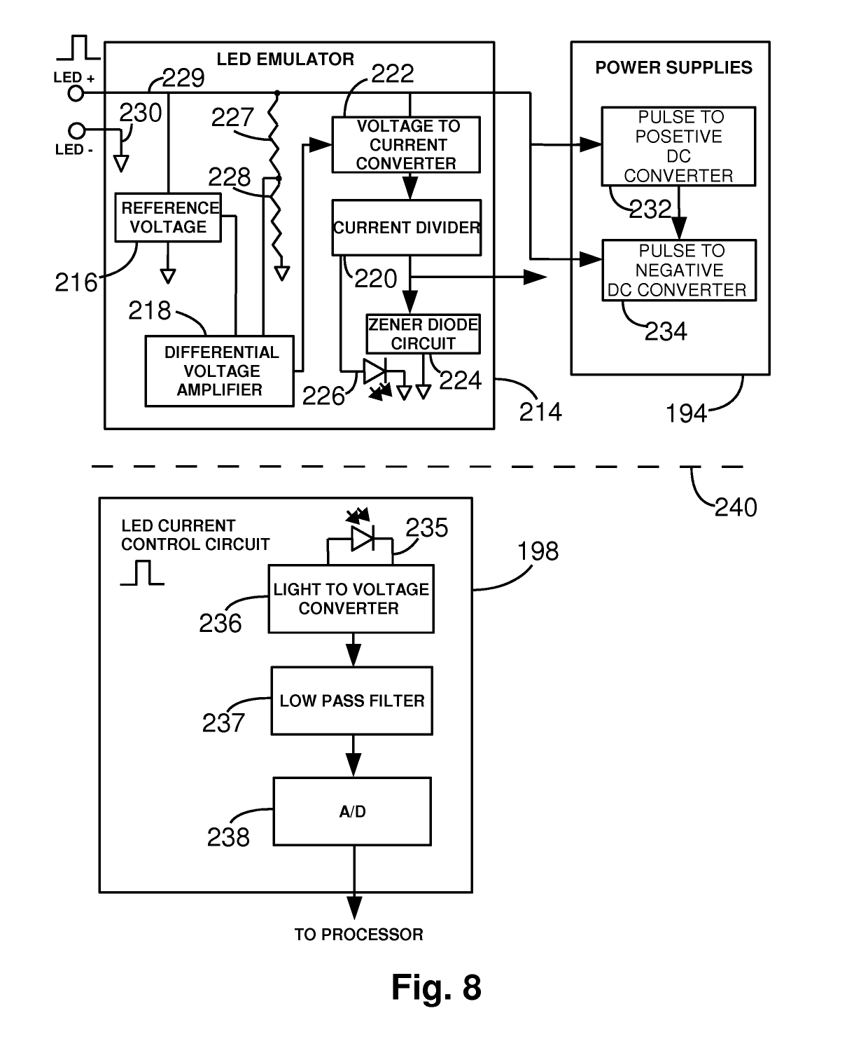

A schematic block diagram of the LED emulator in accordance with some embodiments of the present invention is descried in FIG. 8 to which reference is now made. LED emulator 214 is energized by current pulses of SpO₂ socket of the medical monitor. The electrical currents coming from the SpO₂ socket are typically current pulses which are used to drive in standard medical prior art the LEDs in the patient side. LED emulator 214 includes reference voltage circuitry 216, differential voltage amplifier 218, current divider 220, voltage to current converter 222, zener diode circuit 224 and LED 226 of LCC. Resistors 227 and 228 are used as voltage dividers. The output voltage signal of reference voltage circuitry 216 is the reference voltage for differential voltage amplifier 218. As long as the voltage divider output is smaller than the voltage reference output, the difference between the voltages is amplified by differential voltage amplifier 218 and amplified voltage is converted to current by voltage to current converter 222 until that the output voltage of reference voltage circuitry 216 is equal to the output voltage of the voltage divider. Current divider 220 divides the current that flows from voltage to current converter 222. Part of the current is used for emitting LED 226 of LCC and the rest of the current flows to zener diode circuit 224 which outputs pulses of voltage in the frequency of current pulse sourced SpO₂ socket of the monitor. This voltage source is connected to the CPPL input, not shown. The voltage source across lines 229,230 is fed to power supply module 194 which includes pulse to positive DC converter 232 and pulse to negative DC converter 234. LCC 198 includes photodiode 235 and light to voltage converter 236 for converting the light pulses to electrical pulses. The LCC further includes low pass filter (LPF) 237 and analogue to digital converter (A/D) 238. The digital data is sent to a processor, not shown, for further processing in order to measure the current that is sent from the SpO₂ socket. Dashed line 240 designates that LED emulator 214 is electrically isolated from LCC 198.

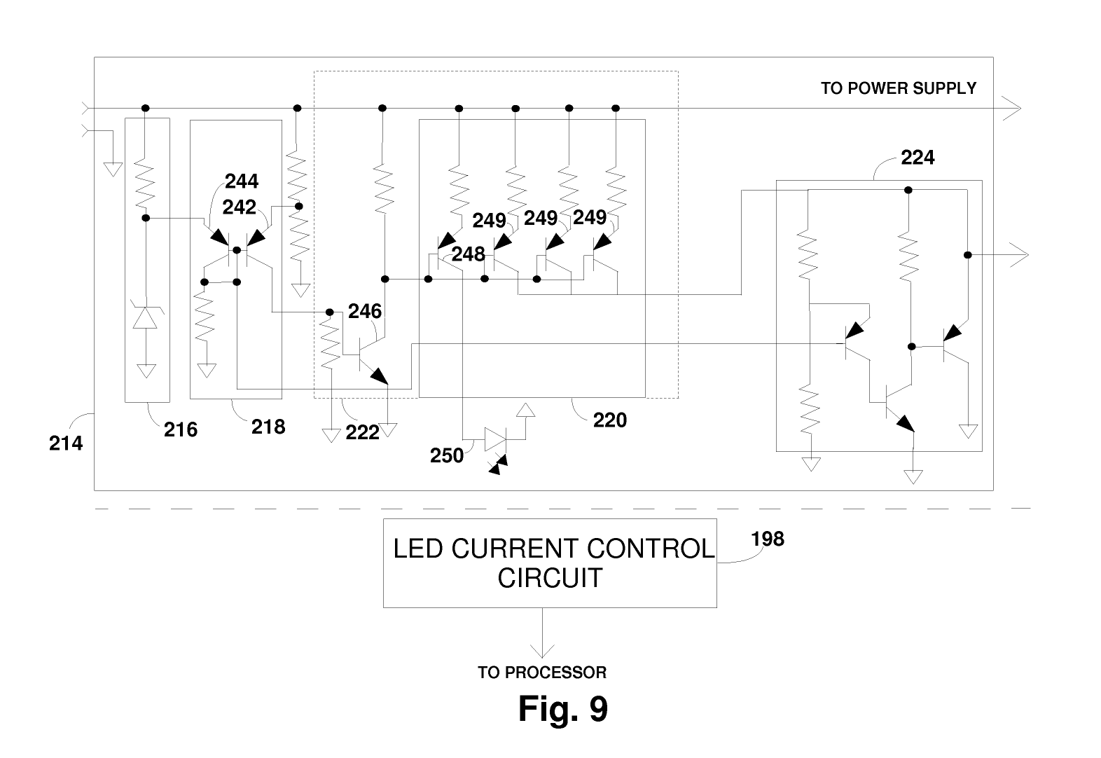

An electronic scheme of LED emulator in accordance with some embodiments of the present invention is described in FIG. 9 to which reference is now made. LED emulator 214 receives current pulse from the monitor. During the front rising section of the pulse, transistors 242 and 244 of differential amplifier 218 increase current into base of transistor 246 thus, current flows through transistors 248 and 249 collector increase and, when the voltage reaches for example to 2.3V the voltage on the LED emulator is stabilized. All circuits of transistors 248 and 249 have identical parameters, so that the currents in these circuits are equal. Therefore, ¼ of all current of transistor 248 flows into LED 250, while ¾ of the current of transistors 249 flows into zener diode circuit 224 that is used to emulate zener diode characteristics but with voltage stabilization accuracy greater than standard diode zener. The output of the zener diode circuit 224 are 2V voltage stabilized pulses which are fed to a converter of pulsative voltage to pulsed light, not shown.

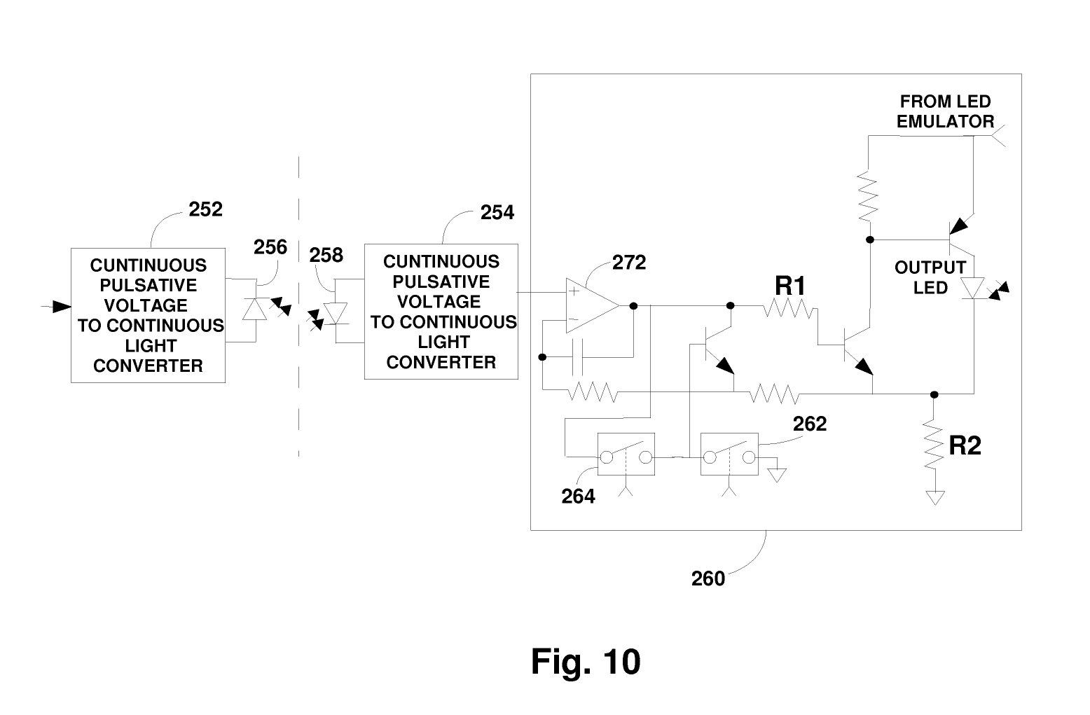

An electronic scheme of isolated continuous pulsative voltage to pulse light converter in accordance with some embodiments of the present invention is described in FIG. 10 to which reference is now made. The continuous voltage to pulsed light converter is restricted in some aspects. Preferably the continuous voltage to pulsed light converter is based on micropower amplifier (an example for such amplifier is TLV2252 of Texas Instruments), because the power supplies of the pulse oximeter emulator are of very low power. The energy supply of the output LED is a voltage pulse. The delay between the voltage pulse front and light pulse front must not be longer than a few microseconds. The continuous electrical signal representing the patient pulsing arterial blood is input to continuous pulsative voltage to continuous light converter 252 which is used to convert voltage to light substantially linearly and to isolate the processor part from SpO₂ socket. Light to continuous pulsative voltage converter 254 is used to convert light to voltage substantially linearly. LED 256 and photodiode 258 in association with converters 252 and 254 optically isolate between continuous pulsative voltage to pulsed light converter circuitry 260 and LPF 206 as shown in FIG. 7.

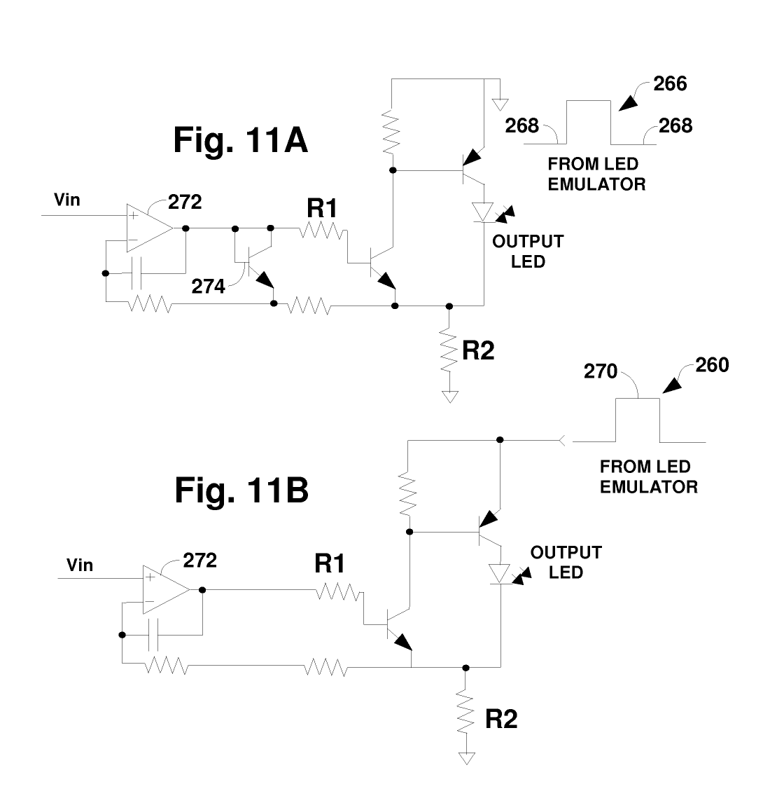

Referring now to FIG. 10 which is an electronic schema of continuous voltage to pulse light converter. Switches 262 and 264 are controlled by a logic circuit that is triggered by the signal pulse that flows from the LED emulator output. When switch 264 is closed and switch 262 is opened, the equivalent electronic scheme is as shown in FIG. 11A. When switch 264 is opened and switch 262 is closed, the equivalent electronic scheme is as shown in FIG. 11B. Referring to FIG. 11A, voltage signal from LED emulator output 266 is in its lowest state 268 and is substantially zero. Referring to FIG. 11B, voltage signal from LED emulator 266 is in its highest state 270 and preferably has a value of 2 Volts.

In order to prevent from micropower amplifier 272 to get into saturation and consequently to prevent light pulse to begin with relatively light overshoot, transistor 274 is connected to the circuit as in equivalent scheme 11A. According to the present invention the amplifier output voltage practically does not change during transition of pulse voltage from low to high and conversely. In FIG. 11A the amplifier output voltage, V_{c}, is approximately V_{c}=V_{be}˜0.6v, and now referring again to FIG. 11B the amplifier output voltage, Vc, is approximately V_{c}=V_{be}+V_{R1}˜0.6V. In these conditions the delay between the voltage pulse front and light pulse front is minimal.

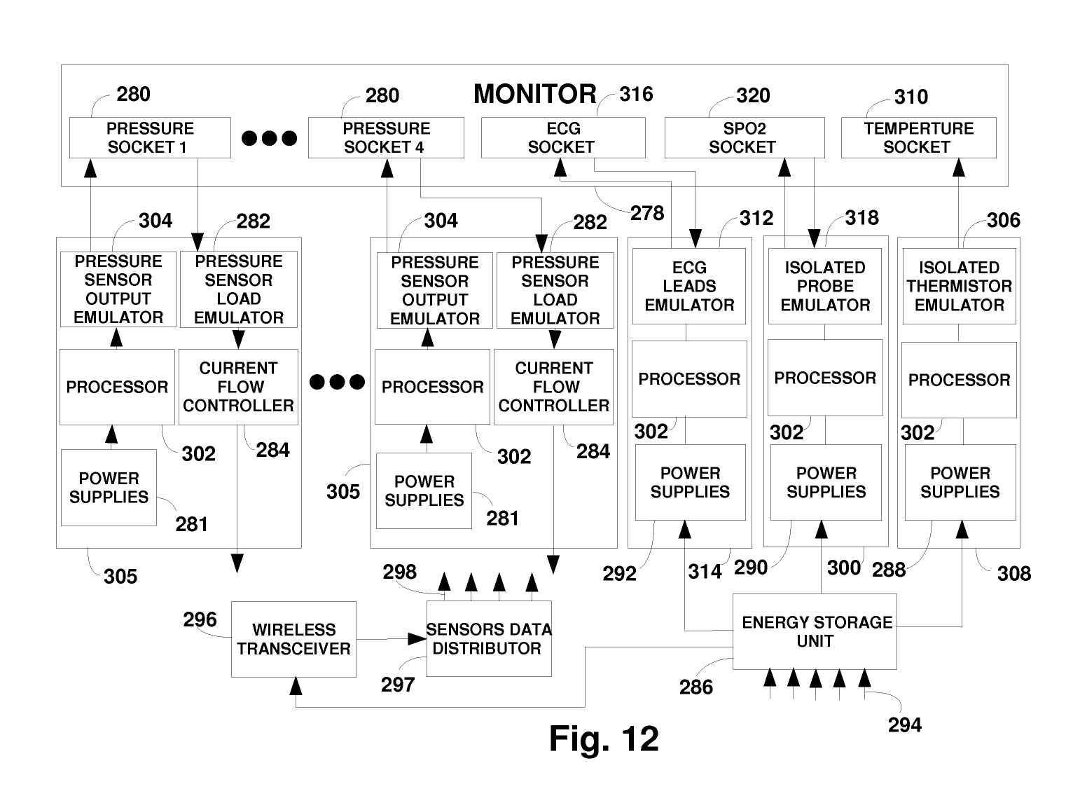

In one aspect of the present invention the monitor wireless transceiver module (MWT) is powered by electrical power partially obtained from pressure sensor sockets of the monitor. A schematic description of the monitor wireless transceiver module (MWT) employed in accordance with one embodiment of the present invention is shown in FIG. 12 to which reference is now made. Monitor 278 includes one or more pressure sensor sockets 280. Pressure sensors sockets 280 of the monitor deliver current to pressure sensor load emulator circuits 282 that emulate the pressure sensor resistance. Current flow controller 284 permits current to flow in one direction towards energy storage unit 286. Such energy storage is typically a capacitor or an accumulator. Current flow controllers 284 are used for supplying power to power supply circuits 288,290 and 292. Arrows 294 designate the energy received from current flow controllers 284. Wireless communications subsystem 296 is used for receiving the wireless digital data transmitted from patient companion assembly (PCA), not shown. This received data includes data collected from PCA subunits, not shown, such as the ECG subunit, SpO₂ subunit, temperature subunit and pressure sensor subunit. Sensors data distributor 297 is used for distributing the sensors data to the respective sensor subunit of the monitor-side. For example, arrow 298 designates the sensor data that further processed in SpO₂ subunit 300 of the monitor-side. The received digital data from Sensors data distributor 297 are further processed in the respective processors 302. Modules 304 of pressure sensor subunits 305 of the monitor-side are used for emulating the signal provided to pressure sockets 280 respectively. Emulator Module 306 of temperature unit 308 of the monitor-side is used for emulating the input signal provided to thermistor socket 310. Emulator Module 312 of ECG unit 314 of the monitor-side is used for emulating the input signal provided to ECG sockets 316. An example of such module is described in FIG. 5 to which reference is again made. Referring back to FIG. 11, Module 318 is used for emulating the input signal provided to SpO₂ sockets 320. An example of such module is described in FIG. 6 to which reference is again made.

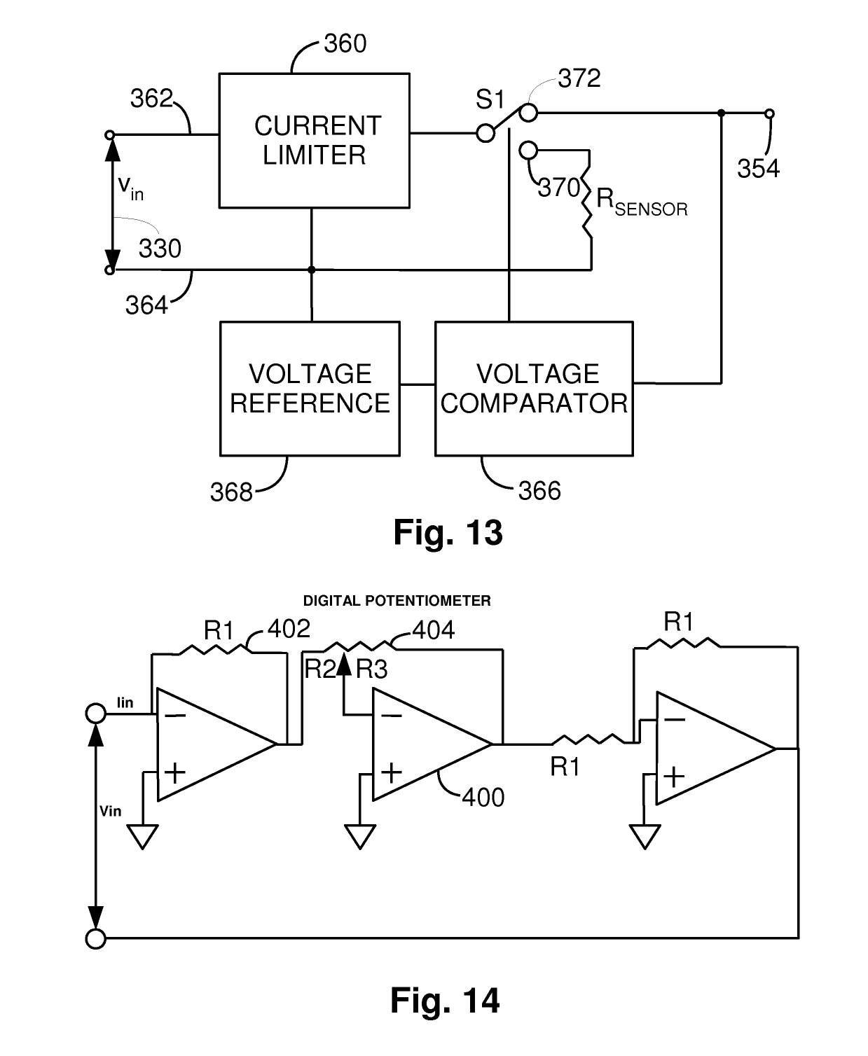

A schematic description of the sensor load emulator and the current flow controller in accordance with some embodiments of the present invention is described in FIG. 13 to which reference is now made. Double headed arrow 330 designates the input voltage received from pressure sensor socket, not shown. Output port 354 is connected to input port 294 of energy storage unit 286. Current limiter 360 limits the current that flows to energy storage unit 294. A relationship between Iₗᵢₘ, Vᵢₙ and Rₛₑₙₛₒᵣ shown in FIG. 13 is given by equation 1 as follows:

Iₗᵢₘ=Vᵢₙ/Rₛₑₙₛₒᵣ (1)

Where Vᵢₙ is the voltage across lines 362 and 364, Rₛₑₙₛₒᵣ is the load emulation of the pressure sensor (preferable value should be minimal with respect to the standard (AAMI BP22) value) , Iₗᵢₘ is the limited current.

Voltage comparator 366 compares between the voltages of the voltage reference 368 and output voltage across port 354. If voltage reference is higher than output voltage across port 354, then comparator 366 commands S1 to switch to port 372 and the storage energy unit 286 is charged. If voltage reference is lower than output voltage across port 354 then, comparator 366 commands S1 to switch to port 370.

Medical Thermistor Emulator

A thermistor is a resistor whose resistance changes with temperature. Because of the known dependence of resistance on temperature, the resistor can be used as a temperature sensor.

Typical medical thermistor accuracy is 0.1° C. A standard medical thermistor changes his resistance from 2252 OHMS at 25° C. to 1023 OHMS at 43° C., which is approximately 4% at each degree. To obtain measurement accuracy better than 0.1° C. it is desirable to achieve the accuracy of the thermistor resistance emulation much better than 0.4%.

A digital potentiometer adjusts and trims electronic circuits similar to variable resistors, rheostats and mechanical potentiometers. These devices can be used to calibrate system tolerances or dynamically control system parameters. A digital potentiometer resistance is usually 10×10³ to 100×10³ [Ohm] with a tolerance of 10%-25%. It is not suitable for the precision emulation of the medical thermistor. However, digital potentiometer working as ratiometric divider has a small temperature coefficient (about 5-35 ppm/° C.) and high linearity. Therefore, it can be exploited as a precision divider for division or multiplication schemes. An electronic scheme of medical thermistor emulator in accordance with the present invention is described in FIG. 14 to which reference is now made. Processor 154 of PCAWT 150 processes the data received from temperature subunit 41 having a thermistor for producing a resistance digital data representing the thermistor resistance. The resistance digital data is wirelessly transmitted through PCAWT 150 to temperature subunit 33 employed in monitor wireless transceiver unit 28 connected to medical monitor unit 30. The emulator of the thermistor of the invention scheme is an analog programmable device, with the following relations between the input current and the input voltage given by equation 2:

Vᵢₙ=Iᵢₙ(R₁(R₃/R₂)) (2)

Where Vᵢₙ is the voltage across the input of the medical thermistor emulator, and Iᵢₙ is the input current of the medical thermistor. Precision resistor R1 402 determines the emulation accuracy. The emulator of the medical thermistor is further includes operational amplifier 400 such as quadruple low-voltage operational amplifier, TLV2254 from Texas Instruments. Digital potentiometer 404 used in a divider mode (R3/R2) that defines the multiplication coefficient and determines the variable thermistor resistance value. Processor 60 receives the resistance digital data and accordingly defines multiplication coefficient such that the emulated resistance which is given by equation 3 represents the resistance represented by the resistance digital data:

Rₑₘᵤₗₐₜₒᵣ=(R₁(R₃/R₂)) (3)

It should be understood that the above description is merely exemplary and that there are various embodiments of the present invention that may be devised, mutatis mutandis, and that the features described in the above-described embodiments, and those not described herein, may be used separately or in any suitable combination; and the invention can be devised in accordance with embodiments not necessarily described above.

1-24. (canceled)

25. A blood oxygen saturation level (SpO₂) measurement subunit employed in a wireless transceiver unit connected to at least one medical monitor unit, said at least one medical monitor unit having at least one SpO₂ socket, said SpO₂ measurement subunit comprising:
an illumination emulator, emulating the characteristics of at least one illumination source of a pulse oximeter;
a processor, employed in said wireless transceiver unit or said SpO₂ measurement subunit, processing information about pulsing arterial blood of a patient received from a patient companion assembly (PCA).

26. A blood oxygen saturation level (SpO₂) measurement subunit employed in a wireless transceiver unit connected to at least one medical monitor unit, said at least one medical monitor unit having at least one SpO₂ socket, said SpO₂ measurement subunit comprising:
at least one power supply circuit supplying energy to electrical components of said SpO₂ measurement subunit.

27. A blood oxygen saturation level (SpO₂) measurement subunit according to claim 26, further comprising:
an illumination emulator, emulating the characteristics of at least one illumination source of a pulse oximeter, wherein said illumination emulator utilises at least part of the energy coming from said at least one SpO₂ socket of said at least one medical monitor unit, said part of the energy originally intended to energise said at least one illumination source of said pulse oximeter, to energise said at least one power supply circuit.

28. A blood oxygen saturation level (SpO₂) measurement subunit according to claim 27, further comprising:
a processor, employed in said wireless transceiver unit or said SpO₂ measurement subunit, processing information about pulsing arterial blood of a patient received from a patient companion assembly (PCA) and providing digitally processed data about said pulsing arterial blood;
a digital to analogue converter converting said digitally processed data into an analogue signal; and
a low pass filter (LPF), filtering said analogue signal,
wherein an output signal of said LPF is a pulsative voltage signal, forming a continuous electrical signal representing the pulsing arterial blood of said patient, and
said pulsative voltage signal is sent to said at least one SpO₂ socket of said at least one medical monitor unit for displaying and further processing.

29. A blood oxygen saturation level (SpO₂) measurement subunit according to claim 26, and also comprising a circuit selected from the group consisting of an IR led circuit and a red led circuit.

30. A blood oxygen saturation level (SpO₂) measurement subunit according to claim 26, wherein said at least one power supply circuit comprises a pulse to positive DC converter and a pulse to negative DC converter.

31. A blood oxygen saturation level (SpO₂) measurement subunit according to claim 28, wherein said illumination emulator includes a current divider for dividing an electrical current coming from said at least one SpO₂ socket.

32. A blood oxygen saturation level (SpO₂) measurement subunit according to claim 31, wherein a first part of an input current of said illumination emulator flows to a continuous pulsative voltage to pulse light converter circuit (CPPL), and a second part of said input current of said illumination emulator flows to a current control circuit.

33. A blood oxygen saturation level (SpO₂) measurement subunit according to claim 32, wherein said continuous pulsative voltage to pulse light converter circuit (CPPL) converts said first part of said input current into pulses of light thereby electrically isolating said at least one SpO₂ socket.

34. A blood oxygen saturation level (SpO₂) measurement subunit according to claim 33, wherein:
said CPPL receives said first part of said input current and said pulsative voltage signal, and modulates pulses of said first part of said input current based on an amplitude of said pulsative voltage signal to provide modulated pulses,
said CPPL utilizes said modulated pulses to cause said illumination source to emit modulated pulses of light, and
a photodiode is connected to said SpO₂ socket and detects the modulated pulses of light emitted from said illumination source.

35. A blood oxygen saturation level (SpO₂) measurement subunit according to claim 34 and also comprising:
at least one photodiode; and
at least one light pulse control circuit, connected to said illumination source,
wherein said at least one photodiode detects the modulated pulses of light emitted from said illumination source, and
said at least one light pulse control circuit and said at least one photodiode are used in association with said processor for insuring that the information about the pulsing arterial blood of a patient is the same as the modulated pulses of light detected by said photodiode connected to said SpO₂ socket.

36. A blood oxygen saturation level (SpO₂) measurement subunit according to claim 33, wherein said current control circuit includes:
at least one photodiode;
a light to voltage converter converting light pulses to electrical pulses;
a low pass filter (LPF); and
an analogue to digital converter (A/D) providing digital data, and
the digital data is sent to said processor for further processing to measure current pulses from said SpO₂ socket for purposes of correct SpO₂ emulation.

37. A blood oxygen saturation level (SpO₂) measurement subunit according to claim 27, wherein said illumination emulator is energized by current pulses of said at least one SpO₂ socket of said at least one medical monitor unit.

38. An electrocardiogram (ECG) monitor subunit employed in association with a patient companion assembly (PCA) in wireless communication with at least one medical monitor unit, said ECG monitor subunit comprising a processor processing ECG data received from the PCA,
said ECG data including one or more measurements for each ECG lead,
said ECG monitor subunit being operative:
to provide said ECG data to a digital to analogue (D/A) converter, said D/A converter providing an analog date output,
to filter the analog data output using a low pass filter, said low pass filter providing a low pass filter output signal, and
to attenuate said low pass filter output signal thereby adapting said low pass filter output signal to a desired intensity level acceptable for input to the at least one medical monitor unit.

39. An electrocardiogram (ECG) subunit employed in a patient companion assembly (PCA) for wireless communication with at least one medical monitor unit, said ECG subunit including a digital wireless communications subsystem, said ECG subunit including a self test generator injecting pulses to test an entire path of ECG data.

40. An electrocardiogram (ECG) subunit employed in a patient companion assembly (PCA) for wireless communication with at least one medical monitor unit, said ECG subunit including a digital wireless communications subsystem providing, to said at least one medical monitor unit, data about one or more disconnected ECG leads.

41. An electrocardiogram (ECG) subunit employed in a patient companion assembly (PCA) wirelessly communicating with at least one medical monitor unit, said ECG subunit processing input arriving from at least two ECG leads, said ECG subunit comprising:
a medical sensor interface subunit having at least two ECG channel routes, each of said at least two ECG channel routes incorporating an ECG channel interface;
an analogue to digital converter;
a multiplexer for multiplexing output signals from said at least two ECG channel routes to said analogue to digital converter;
a digital wireless communications subsystem (WSS) wirelessly communicating with a monitor wireless transceiver unit (MWT); and
a processor for adapting a digital output from said analogue to digital converter to digital wireless communications for supplying to said digital wireless communications subsystem,
said multiplexer multiplexing said output signals in at least two different sequences to compensate for a phase shift between said at least two ECG channel routes.

42. An electrocardiogram (ECG) subunit according to claim 41 wherein said medical sensor interface subunit further comprises:
a defibrillator protection circuit receiving an input from at least one ECG electrode having at least one ECG lead and providing an output signal;
a preamplifier amplifying said output signal of said defibrillator protection circuit and providing a preamplifier output signal;
a lead-off detector receiving said preamplifier output signal, said lead-off detector confirming that an ECG lead connection to a body of a patient is intact;
a band pass filter and amplifying unit receiving said preamplifier output signal and providing an amplifier output signal;
an analogue to digital (A/D) converter, converting said amplifier output signal to digital data; and
a pacemaker detector receiving said preamplifier output signal and providing a pacemaker signal presence output to said processor.

43. An electrocardiogram (ECG) subunit according to claim 42 wherein said preamplifier is a low noise amplifier (LNA).

44. An electrocardiogram (ECG) subunit according to claim 42, wherein said band pass filter and amplifying unit includes a band pass filter in the frequency range of 0.05 Hz-300 Hz.

45. An electrocardiogram (ECG) subunit according to claim 41 wherein said wireless communications subsystem communicates data about one or more disconnected ECG leads to said monitor wireless transceiver unit (MWT), said monitor wireless transceiver unit selecting a connected lead as a reference lead and communicating said reference lead to said PCA.

46. An electrocardiogram (ECG) subunit according to claim 41 and also comprising a self test generator injecting test pulses to test an entire path of at least one of said at least two ECG channel routes.

47. An electrocardiogram (ECG) subunit according to claim 41 and also comprising an electrical circuit for filtering out a frequency of network power.

48. A system for powering a wireless transceiver module connected to a medical monitor having at least one pressure sensor socket, said system powering said wireless transceiver module by an electrical power partially obtained from pressure sensor sockets of a medical monitor, said system comprising:
a pressure sensor load emulator circuit emulating an electrical resistance of a pressure sensor connected to a pressure socket of said medical monitor.

49. A system for powering a wireless transceiver module according to claim 48 and further comprising:
an energy storage unit supplying power to said wireless transceiver module; and
a current flow controller connected to said energy storage unit permitting current flow in one direction towards said energy storage unit.

50. A system for powering a wireless transceiver module according to claim 49 wherein said energy storage unit is an accumulator.

51. A system for powering a wireless transceiver module according to claim 49 wherein said energy storage unit is a super-cap.

52. A system for powering a wireless transceiver module according to claim 49 wherein said current flow controller comprises a current limiter limiting current flowing to said energy storage unit.

53. A system for powering a wireless transceiver module according to claim 52 wherein said current limiter calculates a current limitation using the equation:
Iₗᵢₘ=Vᵢₙ/Rₛₑₙₛₒᵣ
where Vᵢₙ is an input voltage received from said at least one pressure sensor socket,
Rₛₑₙₛₒᵣ is a load emulation resistance value of said pressure sensor, and
Iₗᵢₘ is said current limitation.

54. An emulator of a medical thermistor for use in a wireless transceiver unit connected to at least one medical monitor unit, said at least one medical monitor unit having at least one temperature socket, said emulator being a programmable device having a digital potentiometer working as a ratiometric divider, said emulator determining a function between entrance voltage and entrance current according to a resistance ratio between R₃ and R₂ as given by the equation:
Vᵢₙ=Iᵢₙ(R₁(R₃/R₂))
where Vᵢₙ is a voltage across an input of said emulator of a medical thermistor,
R₁ is a precision resistor determining thermistor emulation accuracy, and
R₃/R₂ is a digital potentiometer ratio used in a divider mode defining a multiplication coefficient (R₃/R₂) and thereby determining a variable thermistor resistance value.

55. A wireless medical monitor comprising:
a wireless monitor transceiver unit; and
a medical monitor unit,
said wireless monitor transceiver unit including a plurality of subunits selected from an ECG subunit, a SpO₂ subunit, a temperature subunit, a pressure subunit, a respiratory subunit and a blood chemistry sub unit,
each said plurality of subunits sharing, with at least one other of said plurality of subunits, at least one of a wireless communication subsystem, a processor, a digital to analogue (D/A) converter, an analogue to digital (A/D) converter, an opto-coupler, a power supply and a multiplexer.
